(19) United States
(12) Patent Application Publication
(10) Pub. No.: US 2002/0110537 A1
(43) Pub. Date: Aug. 15, 2002

(54) Method of and a material for minimizing Rhus dermatitis

(76) Inventor: James Winchester, McLean, VA (US)

Correspondence Address: Ilya Zborovsky, 6 Schoolhouse Way, Dix Hills, NY, 11746, US

(21) Appl. No.: 09/782,009

(22) Filed: Feb. 14, 2001

(51) Int. Cl. A61K 31/785

(52) U.S. Cl. 424/78.35

(57) ABSTRACT

For minimizing Rhus dermatitis a material is used which is topically on a skin and includes an which adsorbs at least a component of an urushiol oil. The material can include of porous hydrophobic divinylbenzene copolymer which initially has surface exposed vinyl groups in which thereafter the vinyl groups are chemically modified so as to form different surface exposed functional groups which hydrophilic and biocompatible.

### BACKGROUND OF THE INVENTION

The present invention relates to a method of and material for minimizing Rhus dermatitis.

Rhus dermatitis is associated with exposure to irritant urushiol oil and other irritating constituents of the sap of the stamps, leaves and roots of poison ivy, poison oak, poison sumac and related plants and plant parts. Urushiol oil itself can contain for example 3-alkylcatechols and 3-alkenylcatechols in which the alkyl or alkenyl moieties are generally 15-17 carbons in length, and compounds such as these can be extremely irritating to the skin.

Various methods and materials are proposed for treatment of the dermatitis. One of the patents, in particular U.S. Pat. No. 3,922,342 discloses also a material for minimizing subsequent dermatitis, which can be applied topically on a skin. The material disclosed in this reference is a hydrophilic ion exchange material which can absorb, upon contact with exposed skin, the phenolic compounds in the urusial oils.

### SUMMARY OF THE INVENTION

Accordingly, it is an object of present invention to provide a new method of minimizing Rhus dermatitis.

In keeping with these objects, one feature of the invention resides in a method which includes the steps of topically applying on a skin a composition including an adsorbent which adsorbs at least one skin irritating component of an urushiol oil.

In accordance with the present invention, the adsorbing material can be composed of porous hydrophobic divinylbenzene copolymer which initially has surface exposed vinyl groups in which thereafter the vinyl groups are chemically modified so as to form different surface exposed functional groups which are hydrophilic and biocompatible.

It is also a feature for present invention to provide a material for minimizing Rhusdermatitis which includes a topically applicable composition with an adsorbent which adsorbs at least one skin irritating component of an urushiol oil.

When the method is performed and the material is formed in accordance with the present invention, at least one or more skin irritating components of the urushiol oil is adsorbed by the new inventive material in accordance with the new inventive method and can prevent Rhus dermatitis.

The novel features which are considered as characteristic for the present invention are set forth in particular in the appended claims.

### DESCRIPTION OF PREFERRED EMBODIMENTS

In accordance with the present invention, to minimize Rhus dermatitis a material is used, which is an adsorbent capable of adsorbing at least one or more skin irritating components of urushiol oil and other irritating constituents of the sap of the stems, leaves and roots of poison ivy, poison oak, poison sumac and related plants and plant parts.

The material can have a size, a shape and a structure selected so as to adsorb said components of the urushiol oil of

The material in accordance with the present invention which is used in the inventive method can be a porous hydrophobic divinylbenzene copolymer which initially has surface exposed vinyl groups in which thereafter the vinyl groups are chemically modified so as to form different surface exposed functional groups with a greater hydrophilicity and greater biocompatibility than those of the vinyl groups, as disclosed for example in U.S. Pat. Nos. 6,087,300; 6,114,466; 6,133,393; 6,136,424, and 6,157,707.

According to the present invention, the modification of the surface vinyl groups was made in aqueous or aqueous organic media by grafting hydrophilic polymer chains by a radial polymerization of 2-hydroxyethyl methacrylate, N-vinylpyrrolidone, N-vinylcaprolactame, or other water soluble monomers, oxidation of the vinyl groups to epoxy groups with the subsequent reaction of the epoxy groups with water, ethylene glycol, amines or 2-amonoethanol molecules, and depositing high-molecular-weight hemocompatible polymer, in particular poly(trifluorethyoxy) phosphazene onto the surface of the polymeric beads.

In any case the hydrophilic nature of thus modified surfaces could be visualized by the easy wetting of dried modification material with water, whereas the initial dry unmodified adsorbent cannot be wetted by an immediate contact with water.

The porous hydrophobic divinylbenzene copolymer can comprise a copolymer divinylbenzene with comonomers selected from the group consisting of styrene, ethylstyrene, acrylo nitrile, buthyl methacrylate.

The surface exposed functional groups which are hydrophilic and biocompatible can be grafted hydrophilic polymeric chains selected from a group which includes polymers of 2-hydroxyethyl methacrylate, N-vinylpyrrolidone, N-vinylcaprolatame, N-acrylamide.

The surface exposed functional groups can be also products of oxidation of said vinyl groups to epoxy groups and subsequent addition of polar compounds selected from a group which includes water, ethylene glycole, small primary or secondary amines, 2-hydroxyethyl-amine.

Also, the surface exposed functional groups can be products of oxidation of said vinyl groups to epoxy groups and subsequent addition of small primary or secondary amines or 2-hydroxyethyl-amine and depositing high-molecular-weight poly(trifluoroethoxy) phosphazene.

The material can be produced by methods which are disclosed in the above mentioned U.S. patents, which incorporated here by means of a reference.

When an absorbent in accordance with the present invention is applied topically on a skin of a person, it adsorbs at least one ore more skin irritating components of Urushiol oil and other irritating constitutes of the sap of the stems, leaves, and roots of poison ivy, poison oak, poison sumach and related plants and plant parts. Therefore, it minimizes rhus dermatitis.

It will be understood that each of the elements described above, or two or more together, may also find a useful application in other types of methods and products differing from the types described above.

While the invention has been illustrated and described as embodied in method of and material for purification of physiological liquids of organism, and method of producing the material it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

We claim:

1. A method of minimizing Rhus dermatitis, comprising the steps of topically applying on a skin a composition including an adsorbent which adsorbs at least a component of an urushiol oil.

2. A method as defined in claim 1, wherein said composition includes porous hydrophobic divinylbenzene copolymer which initially has surface exposed vinyl groups in which thereafter the vinyl groups are chemically modified so as to form different surface exposed functional groups which hydrophilic and biocompatible.

3. A method as defined in claim 2, wherein said porous hydrophobic divinylbenzene copolymer comprises acopolymer of divinylbenzene with comonomers selected from the group consisting of styrene, ethylstyrene, acrylo nitrile, buthyl methacrylate.

4. A method as defined in claim 2, wherein said surface exposed functional groups which are hydrophilic and biocompatible are grafted hydrophilic polymeric chains selected from a group which includes polymers of 2-hydroxyethyl methacrylate, N-vinylpyrrolidone, N-vinylcaprolactame, N-acrylamide.

5. A method as defined in claim 2, wherein said surface exposed functional groups which are hydrophilic and greater biocompatible are products of oxidation of said vinyl groups to epoxy groups and subsequent addition of polar compounds selected from a group which includes water, ethylene glycole, small primary or secondary amines, 2-hydroxyethyl-amine.

6. A method as defined in claim 2, wherein said surface exposed functional groups which are hydrophilic and greater biocompatibile are products of oxidation of said vinyl groups to epoxy groups and subsequent addition of small primary or secondary amines or 2-hydroxyethyl-amine and depositing high-molecular-weight poly(trifluoroethoxy) phosphazene.

7. A material for minimizing Rhus dermatitis, comprising a composition which is topically applicable on a skin and includes an adsorbent which adsorbs at least a component of an urushiol oil.

8. A method as defined in claim 1, wherein said composition includes porous hydrophobic divinylbenzene copolymer which initially has surface exposed vinyl groups in which thereafter the vinyl groups are chemically modified so as to form different surface exposed functional groups which hydrophilic and biocompatible.

9. A material as defined in claim 8, wherein said porous hydrophobic divinylbenzene coplymer comprises a copolymer of divinylbenzene with comonomers selected from the group which includes styrene, ethylstyrene, acrylo nitrile, buthyl methacrylate.

10. A material as defined in claim 8, wherein said surface exposed functional groups which are hydrophilic and biocompatible are grafted hydrophilic polymeric chains selected from a group which includes polymers of 2-hydroxyethyl methacrylate, N-vinylpyrrolidone, N-vinyicaprolactame, N-acrylamide.

11. A material as defined in claim 8, wherein said surface exposed functional groups which are hydrophilic and greater biocompatible are products of oxidation of said vinyl groups to epoxy groups and subsequent addition of polar compounds selected from a group which includes water, ethylene glycole, small primary or secondary amines, 2-hydroxyethyl-amine.

12. A material as defined in claim 8, wherein said surface exposed functional groups which are hydrophilic and greater biocompatibile are products of oxidation of said vinyl groups to epoxy groups and subsequent addition of small primary or secondary amines or 2-hydroxyethyl-amine and depositing high-molecular-weight poly(trifluoroethoxy) phosphazene.
